(19) United States
(12) Patent Application Publication
(10) Pub. No.: US 2005/0240081 A1
(43) Pub. Date: Oct. 27, 2005

(54) Laryngoscope blade

(75) Inventor: Isaac Eliachar, Indian Wells, CA (US)

Correspondence Address: TAROLLI, SUNDHEIM, COVELL & TUMMINO L.L.P.;SUITE 1111, 526 SUPERIOR AVENUE, CLEVELAND, OH, 44114-1400, US

(73) Assignee: The Cleveland Clinic Foundation

(21) Appl. No.: 11/112,378

(22) Filed: Apr. 22, 2005

Related U.S. Application Data
(60) Provisional application No. 60/564,335, filed on Apr. 22, 2004.

(51) Int. Cl. A61B 1/267

(52) U.S. Cl. 600/199; 600/195

(57) ABSTRACT

A laryngoscope blade for detachably connecting to a handle has a rigid skeleton to resist deformation or breakage. The blade includes a first end and a second opposite end. The first end is connectable to the handle. The second end is insertable through a patient's mouth and down the throat and/or hypopharynx to expose the voice box. A light conductor extends through the blade and transmits light from the handle to the second end. The blade has first and second surfaces extending between the first and second ends. The first surface faces and effectively diverts a patient's tongue when the second end is inserted into the patient's mouth and throat. A resilient cushion extends from the second surface. The cushion is supple and deformable to absorb pressure exerted on the cushion by inadvertent contact with the teeth while being inserted in the patient's mouth and throat. The cushion includes an outer skin with a relatively low coefficient of friction. The cushion helps to prevent or minimize accidental damage to the teeth with no restriction on the insertion of the blade.

### RELATED APPLICATION

This application claims the benefit of U.S. Provisional Application No. 60/564,335, which was filed on Apr. 22, 2004 and is incorporated herein by reference.

### TECHNICAL FIELD

The present invention relates to a laryngoscope, and more specifically, to a laryngoscope blade for detachably connecting to a handle.

### BACKGROUND OF THE INVENTION

A laryngoscope incorporates an interchangeable set of blades that are connected to a single handle that houses batteries and a light source. Any one of the blades may be inserted into a patient's mouth and throat to illuminate and expose the voice box and allow an intubation tube to be guided into the windpipe of the patient. The laryngoscope includes a handle and a variety of interchangeable laryngoscope blades. The blade is made of a rigid skeleton and is detachably connected to the handle. Light is conducted from the handle through a connecting end of the blade to an opposite advancing end of the blade. During insertion of the laryngoscope blade into the throat or pharynx via the mouth, a first surface of the laryngoscope blade is used to engage a patient's tongue to divert and deflect it to expose the larynx and facilitate insertion of the tube into the windpipe. A second surface of the blade is prone to engage the upper incisor teeth of the patient. The teeth of the patient may be chipped, broken, or knocked out due to engagement with the hard surface of the rigid blade. It is known to connect a soft cushion to the blade to protect the teeth of the patient. However, the teeth may sink or dig into the cushion and hinder effortless insertion of the blade into the patient's mouth and throat.

### SUMMARY OF THE INVENTION

A laryngoscope blade for detachably connecting to a handle includes a first end and a second opposite end. The first end is connectable to the handle. The second end is insertable into a patient's mouth. The blade has first and second surfaces extending between the first and second ends. The first surface faces a patient's tongue when the second end is inserted into the patient's mouth. A resilient cushion extends from the second surface. The cushion is deformable to absorb pressure exerted on the cushion while being inserted in the patient's mouth. The cushion includes an outer skin with a relatively low coefficient of friction. Accordingly, the cushion protects the teeth of the patient from being damaged while allowing the blade to be easily inserted into the patient's mouth.

In accordance with one feature of the present invention, the laryngoscope blade is detachably connected to a handle having an actuatable light source that is actuated by the connection of the blade to the handle. The laryngoscope blade includes a main body portion made of a rigid plastic material. The main body portion includes the first and second ends and the first and second surfaces. The first end includes means for connecting to the handle and to the actuatable light source in the handle. The main body portion further includes a flange projecting from the second surface and extending between the first and second ends. The flange includes means for conducting light from the first end toward the second end. The flange is covered at least partially by a resilient cushion layer made of a compliant plastic. The cushion layer allows the flange to deform and absorb pressure exerted on the flange by the upper teeth in the patient's mouth to thereby protect against damage to the upper teeth when the second end is inserted into the patient's mouth. The cushion layer of the flange includes an outer skin made of a compliant plastic with a relatively low coefficient of friction to assist in allowing the upper teeth to slide along the outer skin of the cushion layer as the second end is being inserted into the patient's mouth.

In accordance with another feature of the present invention, the laryngoscope blade includes a surface extending between the first and second ends. The surface is engageable with a patient's tongue when the second end is inserted into the patient's mouth. The surface includes a textured portion engageable with the patient's tongue to help prevent or resist movement of the tongue relative to the blade in a direction extending transverse to a longitudinal extent of the blade.

In accordance with another feature of the present invention, the laryngoscope blade includes a concave surface extending from the first end toward the second end for engaging an intubation tube to guide movement of the intubation tube relative to the blade.

In accordance with another feature of the present invention, the laryngoscope blade includes light conducting means extending toward the second end. The light conducting means including first and second light emitting portions.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is an exploded view of a laryngoscope including a handle and a laryngoscope blade constructed in accordance with a first embodiment;

FIG. 2 is a pictorial view of the laryngoscope blade of FIG. 1;

FIG. 3 is a side view of the laryngoscope blade of FIG. 1;

FIG. 4 is a cross-sectional view of the laryngoscope blade taken along the line 4-4 in FIG. 3;

FIG. 5 is a side view of a laryngoscope blade constructed in accordance with a second embodiment;

FIG. 6 is a side view of a laryngoscope blade constructed in accordance with a third embodiment; and

FIG. 7 is a cross-sectional view of the laryngoscope blade taken along the line 7-7 in FIG. 6.

### DESCRIPTION OF EMBODIMENTS

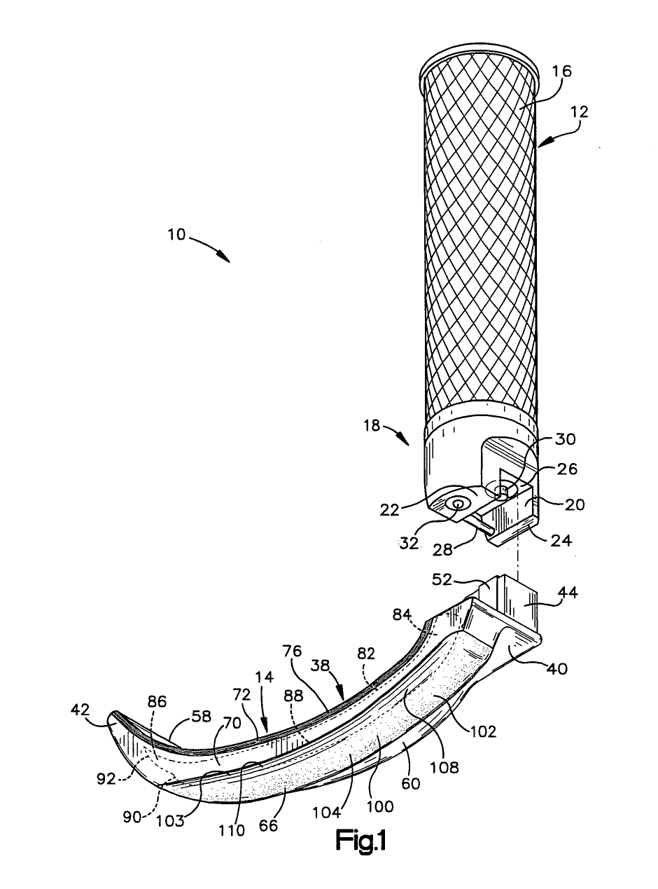

The present invention is directed to a laryngoscope, and more specifically, to a laryngoscope blade. As representative of the present invention, FIG. 1 schematically illustrates a laryngoscope 10 having a handle 12 and a laryngoscope blade 14. The blade 14 is detachably connected to the handle 12. The blade 14 extends at approximately 90° from the handle 12 when the blade is connected to the handle in a locked or 90° snapped in position. The handle 12 may be made of any suitable material, such as metal or a rigid plastic material. It is contemplated that the blade 14 may be disposable and the handle 12 reusable.

The handle 12 has a first upper end 16, as viewed in FIG. 1, having a textured outer surface. The textured outer surface allows the handle 12 to be easily grasped. The upper end 16 contains a power source (not shown), such as a battery, as known in the art. It is contemplated that the power source may be any suitable power source.

The handle 12 includes a second lower end 18, as viewed in FIG. 1, that is connectable with the blade 14. The second end 18 includes a recess 20 for receiving the blade 14. The recess 20 is at least partially defined by generally parallel walls 22 and 24 extending downwardly, as viewed in FIG. 1, from the handle 12. A surface 26 extends between the walls 22 and 24 to further define the recess 20.

A rod 28 extends between the walls 22 and 24. The rod 28 is fixedly connected to the walls 22 and 24 in any suitable manner. The rod 28 extends between lower ends of the walls 22 and 24. The rod 28 is spaced from the surface 26.

A switch 30 is located in the recess 20. The switch 30 extends through the surface 26 into the recess 20. The blade 14 engages the switch 30 to activate a light source 32 when the blade is connected to the handle 12 in the locked or 90° snapped in position. The blade 14 releases the switch 30 when the blade is not in the 90° snapped in position or is disconnected from the handle 12 to deactivate the light source 32. The light source 32 is located in the wall 22 of the handle 12. The light source 32 may be any suitable light source, such as a light bulb. It is contemplated that the light source 32 may be activated and deactivated in any suitable manner. It is also contemplated that the light source 32 may be located in the blade 14.

The blade 14 (FIGS. 1-3) includes an arcuate main body portion or skeleton 38 having a first end 40 and a second opposite end 42. The first end 40 is detachably connectable to the handle 12. The second end 42 is insertable into a patient's mouth. The main body portion 38 may be made of any suitable rigid material, such as a rigid plastic material. It is contemplated that the blade 14 may be disposable after use.

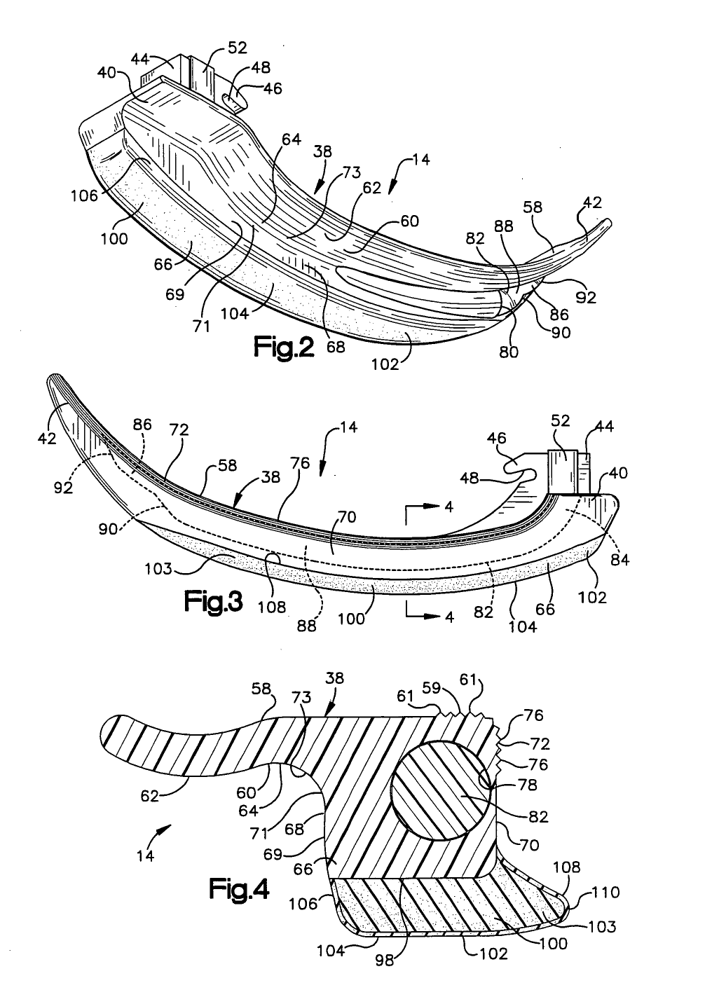

The first end 40 of the blade 14 includes a rectangular projection 44 that is received in the recess 20 in the handle 12 when the blade is connected to the handle. A hook 46 (FIGS. 2 and 3) extends from the projection 44 toward the second end 42 of the blade 14. The hook 46 defines a recess 48 in the second end 40 for receiving the rod 28 when the blade 14 is connected to the handle 12.

A shim portion 52 (FIGS. 1-3) of the blade 14 extends around the rectangular projection 44 in a direction transverse to the longitudinal extent of the main body portion 38. The shim portion 52 engages the walls 22 and 24 of the handle 12 to create an interference fit between the first end 40 of the blade 14 and the handle 12. The shim portion 52 also engages the switch 30 to activate the light source 32 when the blade 14 is connected to the handle 12.

The blade 14 is connected to the handle 12 by inserting the rod 28 into the recess 48 defined by the hook 46 on the blade 14. The blade 14 is then pivoted about the rod 28 to pivot the rectangular projection 44 into the recess 20 in the handle 12. The shim portion 52 engages the walls 22 and 24 of the handle 12 to create an interference fit between the blade 14 and the handle. The shim portion 52 also engages the switch 30 to activate the light source 32. Accordingly, the blade 14 is securely connected to the handle 12. The blade 14 is disconnected from the handle 12 by overcoming the interference fit between the projection 44 and the second end 18 of the handle 12. The blade 14 releases the switch 30 to deactivate the light source 32.

The blade 14 may include a recess (not shown) for receiving a ball bearing (not shown) on the handle 12. The ball bearing snaps into the recess on the blade 14 to secure the blade to the handle in the 90° snapped in position. At the same time, the light source 32 is activated. The blade 14 may swing away from the 90° snapped in position and remain detachably connected to the handle 12. When the blade 14 is not in the 90° snapped in position, the light source 32 is deactivated. The blade 14 may hang from the handle 12 and extend generally parallel to the handle with the light source 32 deactivated. It is contemplated that the blade 14 may be detachably connected to handle 12 in any suitable manner.

The main body portion 38 (FIGS. 1-4) of the blade 14 includes a first upper arcuate surface 58 extending between the ends 40 and 42. The first surface 58 faces and/or engages a patient's tongue when the second end 42 is inserted and advanced into the patient's mouth. The first surface 58 (FIG. 4) has a textured or serrated portion 59 extending from the first end 40 to the second end 42 of the blade 14. The textured or serrated portion 59 includes a plurality of longitudinally extending ribs 61. The textured or serrated portion 59 may gently or atraumatically engage the patient's tongue to help prevent or resist movement or slippage of the tongue relative to the blade 14 in a direction extending transverse or at a right angle to the longitudinal extent of the blade. The textured or serrated portion 59 does not hinder insertion of the blade 14 into the patient's mouth while engaging the tongue. The textured or serrated portion 59 helps divert the tongue to expose the larynx.

A second lower arcuate surface 60 (FIGS. 1-2) of the blade 14 extends between the ends 40 and 42. The second surface 60 faces toward the oropharyngeal cavity and the upper teeth of the patient when the second end 42 is inserted into the patient's mouth. The second surface 60 (FIG. 4) includes a convex portion 62 and a concave portion 64. The concave portion 64 engages an intubation tube to help guide insertion of the intubation tube into the patient's throat, pharynx and/or hypopharynx.

An arcuate flange or ridge 66 projects downwardly, as viewed in FIG. 1, from the second surface 60. The flange 66 extends from the first end 40 of the blade 14 to the second end 42. The flange 66 (FIG. 4) includes a first side surface 68 extending from the surface 60. The side surface 68 includes a convex portion 69 and a concave portion 71. The concave portion 71 of the surface 68 and the concave portion 64 of the surface 60 define a concave cylindrical trough 73. The trough 73 engages the intubation tube to help guide insertion of the intubation tube into the patient's throat.

A second side surface 70 (FIG. 4) of the flange 66 extends from the textured or serrated portion 59 of the first surface 58 on a side of the flange opposite from the side surface 68. The second side surface 70 extends generally perpendicular to the first upper surface 58. The side surface 70 has a textured or serrated portion 72 extending from the first end 40 to the second end 42 of the blade 14. The textured or serrated portion 72 is located adjacent to the textured or serrated portion 59 on the first surface 58 of the blade 14. The textured or serrated portion 72 includes a plurality of longitudinally extending ribs 76. The textured or serrated portion 72 may engage the patient's tongue to help prevent or resist movement or slippage of the tongue relative to the blade 14 in a direction extending transverse or at a right angle to the longitudinal extent of the blade. The textured or serrated portion 72 does not hinder insertion of the blade 14 into the patient's mouth while atraumatically engaging the tongue. The textured or serrated portion 72 helps divert the tongue to expose the larynx.

The flange 66 includes a generally arcuate passage or tunnel 78. The passage 78 extends through the flange 66 from the first end 40 toward the second end 42. The side surface 68 (FIG. 2) has an opening 80 that intersects the passage 78. The opening 80 is located adjacent to the surface 60 approximately ⅔ of the length of blade 14 away from the first end 40. The passage 78 extends from the first end 40 of the blade 14 adjacent the light source 32 when the blade is connected to the handle 12 in the right angle locked position.

A light conductor 82 (FIGS. 1-4) extends through the passage 78. The light conductor 82 conducts or transmits light from the light source 32 toward the second end 42 of the blade 14 to illuminate the patient's throat. The light conductor 82 has a first end 84 located in the first end 40 of the blade 14 adjacent to or facing the light source 32 when the blade is connected to the handle 12. The light conductor 82 has a second end 86 located adjacent the second end 42 of the blade 14. A central portion 88 of the light conductor 82 extends between the first and second ends 84 and 86.

The central portion 88 (FIG. 2) of the light conductor 82 extends through the opening 80 in the flange 66 and includes a first light emitting portion or surface 90. The second end 86 of the light conductor 82 extends from the light emitting portion or surface 90 toward the second end 42 of the blade 14. The second end 86 of the light conductor 82 includes a second light emitting portion or surface 92. It is contemplated that blade 14 may include any suitable light conductor 82, such as a prismatic light conductor or a fiber optic light conductor.

The flange 66 (FIG. 4) has a lower surface 98. The lower surface 98 of the flange 66 is covered at least partially by a resilient cushion or cushion layer 100. The cushion or cushion layer 100 may be made of a compliant plastic and/or filled with a suitable gas or liquid such as air or water. The cushion 100 extends downwardly, as viewed in FIG. 4, from the lower surface 98 of the flange 66. The cushion or cushion layer 100 allows the flange 66 to deform and absorb pressure exerted on the flange by contact with the upper teeth in the patient's mouth to thereby protect against potential inadvertent or accidental damage to the upper teeth when the second end 42 is inserted into the patient's mouth. The cushion 100 is formed as one-piece with the main body portion 38, such as by molding. It is contemplated that the cushion 100 may be fixedly connected to the surface 98 of the flange 66 in any suitable manner, such as by fusing.

The cushion or cushion layer 100 of the flange 66 includes an outer skin 102 made of a slick or slippery compliant plastic. The outer skin 102 has a relatively low coefficient of friction to assist in allowing the upper teeth of the patient to glide or slide effortlessly along the outer skin of the cushion 100 as the second end 42 is being inserted into the patient's mouth. The cushion or cushion layer 100 includes a projection 103 extending from the side surface 70 of the flange 66. The projection 103 of the cushion 100 extends from the side surface 70 in a direction away from the side surface 68 of the flange 66.

The cushion or cushion layer 100 (FIG. 4) includes a lower arcuate surface 104 extending generally parallel to the first surface 58 of the blade 14. The cushion 100 includes a side surface 106 extending from the surface 104 toward the surface 98 of the flange 66. The surface 106 of the cushion 100 extends at an angle from the surface 68 of the flange 66 to the surface 104. The projection 103 includes a surface 108 facing away from the surface 104. A rounded surface 110 extends between the surfaces 104 and 108. The surfaces 106 and 108 blend or merge with the surfaces 68 and 70 of the flange 66 along the length of the blade 14.

FIG. 5 illustrates a laryngoscope blade 114 constructed in accordance with an alternate configuration. In the embodiment of FIG. 5, reference numbers that are the same as those used in the first embodiment of FIGS. 1-4 designate parts that are the same as parts in the first embodiment.

According to the second embodiment, the laryngoscope blade 114 includes a generally straight main body portion or skeleton 138 having a first end 140 and a second opposite end 142. The first end 140 is detachably connectable to the handle 12 shown in FIG. 1. The second end 142 (FIG. 5) is insertable into a patient's mouth. The main body portion 138 may be made of any suitable rigid material, such as a rigid plastic material. It is contemplated that the blade 114 may be disposable after use.

The blade 114 is connected to the handle 12 by inserting the rod 28 into a recess 48 defined by a hook 46 on the blade 114. The blade 114 is then pivoted about the rod 28 to pivot a rectangular projection 44 into the recess 20 in the handle 12. A shim portion 52 of the blade 114 engages the walls 22 and 24 of the handle 12 to create an interference fit between the blade and the handle. The shim portion 52 also engages the switch 30 to activate the light source 32. Accordingly, the blade 114 is securely connected to the handle 12. The blade 114 is disconnected from the handle 12 by overcoming the interference fit between the projection 44 and the second end 18 of the handle 12. It is contemplated that the blade 114 may be detachably connected to the handle 12 in any suitable manner.

The main body portion 138 of the blade 114 includes a first upper surface 158 extending between the ends 140 and 142. The first surface 158 faces and/or engages a patient's tongue when the second end 142 is inserted and advanced into the patient's mouth. The first surface 158 may have a textured or serrated portion (not shown) extending from the first end 140 to the second end 142 of the blade 114. The textured or serrated portion may engage the patient's tongue to help prevent movement or slippage of the tongue relative to the blade 114 in a direction transverse to the longitudinal extent of the blade. The textured or serrated portion helps divert the tongue to expose the larynx.

A second lower surface 160 of the blade 114 extends between the ends 140 and 142. The second surface 160 faces toward the oropharyngeal cavity and the upper teeth of the patient when the second end 142 is inserted into the patient's mouth. The second surface 160 includes a convex portion and a concave portion. The concave portion engages an intubation tube to help guide insertion of the intubation tube into the patient's throat, pharynx and/or hypopharynx.

A flange or ridge 166 projects downwardly, as viewed in FIG. 5, from the second surface 160. The flange 166 extends from the first end 140 of the blade 114 to the second end 142. The flange 166 includes a first side surface 168 extending from the surface 160. The side surface 168 includes a convex portion 169 and a concave portion 171. The concave portion 171 of the surface 168 and the concave portion of the surface 160 define a concave cylindrical trough 173. The trough 173 engages the intubation tube to help guide insertion of the intubation tube into the patient's throat.

The flange 166 includes a passage or tunnel extending through the flange from the first end 140 toward the second end 142. The side surface 168 has an opening 180 that intersects the passage. The opening 180 is located adjacent to the surface 160 approximately ⅔ of the length of blade 114 away from the first end 140. The passage extends from the first end 140 adjacent the light source 32 when the blade 114 is connected to the handle 12 in a right angle locked position.

A light conductor 182 extends through the passage in the flange 166. The light conductor 182 conducts or transmits light from the light source 32 toward the second end 142 of the blade 114 to illuminate the patient's throat. The light conductor 182 has a first end 184 located in the first end 140 of the blade 114 adjacent to or facing the light source 32 when the blade is connected to the handle 12. The light conductor 182 has a second end 186 located adjacent the second end 142 of the blade 114. A central portion 188 of the light conductor 182 extends between the first and second ends 184 and 186.

The central portion 188 of the light conductor 182 extends through the opening 180 in the flange 166 and includes a first light emitting portion or surface 190. The second end 186 of the light conductor 182 extends from the light emitting portion or surface 190 toward the second end 142 of the blade 114. The second end 186 of the light conductor 182 includes a second light emitting portion or surface 192. It is contemplated that the blade 114 may include any suitable light conductor 182, such as a prismatic light conductor, or a fiber optic light conductor.

The flange 166 has a lower surface 198. The lower surface 198 of the flange 166 is covered at least partially by a resilient cushion or cushion layer 200. The cushion or cushion layer 200 may be made of a compliant plastic and/or filled with a suitable gas or liquid such as air or water. The cushion 200 extends downwardly, as viewed in FIG. 5, from the surface 198 of the flange 166. The cushion or cushion layer 200 allows the flange 166 to deform and absorb pressure exerted on the flange by contact with the upper teeth in the patient's mouth to thereby protect against potential inadvertent or accidental damage to the upper teeth when the second end 142 is inserted into the patient's mouth. The cushion 200 is formed as one-piece with the main body portion 138, such as by molding. The cushion 200 may be fixedly connected to the surface 198 in any suitable manner, such as by fusing. The cushion or cushion layer 200 of the flange 166 includes an outer skin 202 made of a slick or slippery compliant plastic. The outer skin 202 has a relatively low coefficient of friction to assist in allowing the upper teeth of the patient to glide or slide effortlessly along the outer skin of the cushion 200 as the second end 142 is being inserted into the patient's mouth.

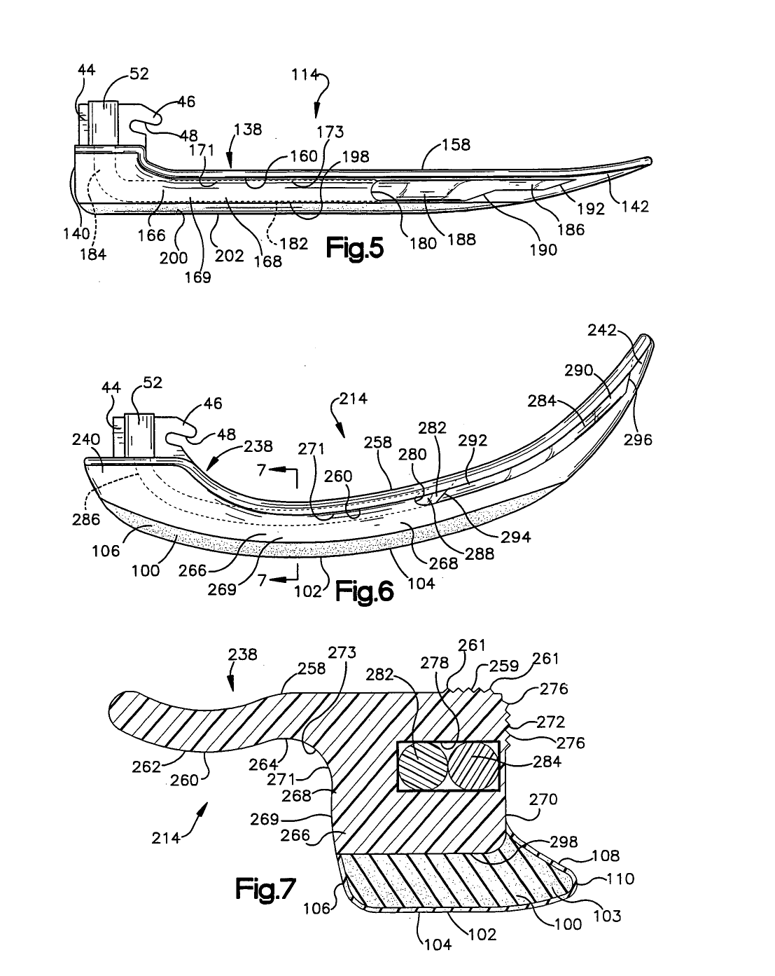

FIGS. 6-7 illustrate a laryngoscope blade 214 constructed in accordance with an alternate configuration. In the embodiment of FIGS. 6-7, reference numbers that are the same as those used in the first embodiment of FIGS. 1-4 designate parts that are the same as parts in the first embodiment.

According to the third embodiment, the laryngoscope blade 214 (FIG. 6) includes an arcuate main body portion or skeleton 238 having a first end 240 and a second opposite end 242. The first end 240 is detachably connectable to the handle 12 shown in FIG. 1. The second end 242 (FIG. 6) is insertable into a patient's mouth. The main body portion 238 may be made of any suitable rigid material, such as a rigid plastic material. It is contemplated that the blade 214 may be disposable after use.

The blade 214 is connected to the handle 12 by inserting the rod 28 into a recess 48 defined by a hook 46 on the blade 214. The blade 214 is then pivoted about the rod 28 to pivot a rectangular projection 44 into the recess 20 in the handle 12. A shim portion 52 of the blade 214 engages the walls 22 and 24 of the handle 12 to create an interference fit between the blade 214 and the handle. The shim portion 52 also engages the switch 30 to activate the light source 32. Accordingly, the blade 214 is securely connected to the handle 12. The blade 214 is disconnected from the handle 12 by overcoming the interference fit between the projection 44 and the second end 18 of the handle 12. It is contemplated that the blade 214 may be detachably connected to the handle 12 in any suitable manner.

The main body portion 238 (FIGS. 6-7) of the blade 214 includes a first upper arcuate surface 258 extending between the ends 240 and 242. The first surface 258 faces and/or engages a patient's tongue when the second end 242 is inserted and advanced into the patient's mouth. The first surface 258 has a textured or serrated portion 259 extending from the first end 240 to the second end 242 of the blade 214. The textured or serrated portion 259 includes a plurality of longitudinally extending ribs 261. The textured or serrated portion 259 may engage the patient's tongue to help prevent or resist movement or slippage of the tongue relative to the blade 214 in a direction extending transverse to the longitudinal extent of the blade. The textured or serrated portion 259 does not hinder insertion of the blade 214 into the patient's mouth while engaging the tongue. The textured or serrated portion 259 helps divert the tongue to expose the larynx.

A second lower arcuate surface 260 of the blade 214 extends between the ends 240 and 242. The second surface 260 faces toward the oropharyngeal cavity and the upper teeth of the patient when the second end 242 is inserted into the patient's mouth. The second surface 260 includes a convex portion 262 and a concave portion 264. The concave portion 264 engages an intubation tube to help guide insertion of the intubation tube into the patient's throat.

An arcuate flange or ridge 266 projects downwardly, as viewed in FIGS. 6 and 7, from the second surface 260. The flange 266 extends from the first end 240 of the blade 214 to the second end 242. The flange 266 (FIG. 7) includes a first side surface 268 extending from the surface 260. The side surface 268 includes a convex portion 269 and a concave portion 271. The concave portion 271 of the surface 268 and the concave portion 264 of the surface 260 define a concave cylindrical trough 273. The trough 273 engages the intubation tube to help guide insertion of the intubation tube into the patient's throat.

A second side surface 270 (FIG. 7) of the flange 266 extends from the textured or serrated portion 259 of the first surface 258 on a side of the flange opposite from the side surface 268. The second side surface 270 extends generally perpendicular to the first upper surface 258. The side surface 270 has a textured or serrated portion 272 extending from the first end 240 to the second end 242 of the blade 214. The textured or serrated portion 272 is located adjacent to the textured or serrated portion 259 on the first surface 258 of the blade 214. The textured or serrated portion 272 includes a plurality of longitudinally extending ribs 276. The textured or serrated portion 272 may atraumatically engage the patient's tongue to help prevent or resist movement or slippage of the tongue relative to the blade 214 in a direction extending transverse to the longitudinal extent of the blade. The textured or serrated portion 272 does not hinder insertion of the blade 214 into the patient's mouth while engaging the tongue. The textured or serrated portion 272 helps divert the tongue to expose the larynx.

The flange 266 includes a generally arcuate passage or tunnel 278. The passage 278 extends through the flange 266 from the first end 240 toward the second end 242. The side surface 268 (FIG. 6) has an opening 280 that intersects the passage 278. The opening 280 is located adjacent to the surface 260 approximately ⅔ of the length of blade 214 away from the first end 240. The passage 278 is located in the first end 240 of the blade 214 adjacent the light source 32 when the blade 214 is connected to the handle 12 in a right angle locked position.

First and second light conductors 282 and 284 extend through the passage 278. The light conductors 282 and 284 conduct or transmit light from the light source 32 toward the second end 242 of the blade 214 to illuminate the patient's throat. Each of the light conductors 282 and 284 has a first end 286, one of which is shown in FIG. 6, located in the first end 240 of the blade 214 adjacent to or facing the light source 32 when the blade is connected to the handle 12. The light conductor 282 has a second end 288 that extends through the opening 280 in the flange 266. The light conductor 284 has a second end 290 located adjacent the second end 242 of the blade 214. A central portion 292 of the light conductor 284 extends through the opening 280 in the flange 266. The second end 288 of the light conductor 282 has a light emitting portion or surface 294. The second end 290 of the light conductor 284 includes a light emitting portion or surface 296. It is contemplated that the blade 214 may include any suitable light conductors 282 and 284, such as prismatic light conductors or fiber optic light conductors.

The flange 266 (FIG. 7) has a lower surface 298. The lower surface 298 of the flange 266 is covered at least partially by a resilient cushion or cushion layer 100. The cushion or cushion layer 100 may be made of a compliant plastic and/or filled with a suitable gas or liquid such as air or water. The cushion 100 extends downwardly as viewed in FIG. 7, from the lower surface 298 of the flange 266. The cushion or cushion layer 100 allows the flange 266 to deform and absorb pressure exerted on the flange by contact with the upper teeth in the patient's mouth to thereby protect against damage to the upper teeth when the second end 242 is inserted into the patient's mouth. The cushion 100 is formed as one-piece with the main body portion 238, such as by molding. The cushion 100 may be fixedly connected to the surface 298 of the flange 266 in any suitable manner, such as by fusing.

The cushion or cushion layer 100 of the flange 266 includes an outer skin 102 made of a slick or slippery compliant plastic. The outer skin 102 has a relatively low coefficient of friction to assist in allowing the upper teeth of the patient to glide or slide effortlessly along the outer skin of the cushion 100 as the second end 242 is being inserted into the patient's mouth. The cushion or cushion layer 100 includes a projection 103 extending from the side surface 270 of the flange 266. The projection 103 of the cushion 100 extends from the side surface 270 in a direction away from the side surface 268 of the flange 266.

The cushion or cushion layer 100 (FIG. 7) includes a lower arcuate surface 104 extending generally parallel to the first surface 258 of the blade 214. The cushion 100 includes a side surface 106 extending from the surface 104 toward the surface 298 of the flange 266. The surface 106 of the cushion 100 extends at an angle from the surface 268 of the flange 266 to the surface 104. The projection 103 includes a surface 108 facing away from the surface 104. A rounded surface 110 extends between the surfaces 104 and 108. The surfaces 106 and 108 blend or merge with the surfaces 268 and 270 of the flange 266 along the length of the blade 214.

Although the laryngoscope blades 14, 114, and 214 are shown as being detachably connected to the handle 12, it is contemplated that the blades may be detachably connected to any suitable handle using a variety of connecting or coupling mechanisms. Although the handle 12 is described as having a light bulb for the light source 32, it is contemplated that the handle may include any suitable light source. It is also contemplated that the handle 12 may include any suitable power source.

From the above description of the invention, those skilled in the art will perceive improvements, changes and modifications. Such improvements, changes and modifications within the skill of the art are intended to be covered by the appended claims.

1. A laryngoscope blade for detachably connecting to a handle having an actuatable light source that is actuated by the connection of said laryngoscope blade to the handle, said laryngoscope blade comprising:
a main body portion made of a rigid plastic material, said main body portion having oppositely disposed first and second ends and first and second surfaces extending between said ends, said first end including means for connecting to the handle and to the actuatable light source in the handle, said second end for insertion into a patient's mouth, said first surface for engaging a patient's tongue when said second end is inserted into the patient's mouth;
said main body portion further including a flange projecting from said second surface and extending between said first and second ends, said flange including means for conducting light from said first end toward said second end;
said flange being covered at least partially by a resilient cushion layer made of a compliant plastic, said cushion layer for allowing said flange to deform and absorb pressure exerted on said flange by the upper teeth in the patient's mouth to thereby protect against damage to the upper teeth when said second end is inserted into the patient's mouth;
said cushion layer of said flange including an outer skin made of a compliant plastic with a relatively low coefficient of friction to assist in allowing the upper teeth to slide along said outer skin of said cushion layer as second end is being inserted into the patient's mouth.

2. A laryngoscope blade for detachably connecting to a handle, said laryngoscope blade comprising:
a first end and a second opposite end, said first end being connectable to the handle, said second end being insertable into a patient's mouth;
first and second surfaces extending between said first and second ends, said first surface facing a patient's tongue when said second end is inserted into the patient's mouth; and
a resilient cushion extending from said second surface, said cushion being deformable to absorb pressure exerted on said cushion while being inserted in the patient's mouth, said cushion including an outer skin with a relatively low coefficient of friction.

3. A laryngoscope blade for detachably connecting to a handle, said laryngoscope blade comprising:
a first end and a second opposite end, said first end being connectable to the handle, said second end being insertable into a patient's mouth; and
a surface extending between said first and second ends, said surface being engageable with a patient's tongue when said second end is inserted into the patient's mouth, said surface including a textured portion engageable with the patient's tongue to help prevent movement of the tongue relative to said blade in a direction extending transverse to a longitudinal extent of said blade.

4. A laryngoscope blade for detachably connecting to a handle, said laryngoscope blade comprising:
a first end and a second opposite end, said first end being connectable to the handle, said second end being insertable into a patient's mouth; and
a concave surface extending from said first end toward said second end for engaging an intubation tube to guide movement of the intubation tube relative to said blade.

5. A laryngoscope blade for detachably connecting to a handle, said laryngoscope blade comprising:
a first end and a second opposite end, said first end being connectable to the handle, said second end being insertable into a patient's mouth; and
light conducting means extending toward said second end, said light conducting means including first and second light emitting portions.
